SEQUENCE LISTING (5 sequences)

SEQ ID NO: 1 - protein, 12 aa, Homo sapiens
AKRHHGYKRKFH

SEQ ID NO: 2 - protein, 12 aa, Homo sapiens
AKRHHGHKRKHH

SEQ ID NO: 3 - protein, 12 aa, Homo sapiens
ALLHHGYKLKFH

SEQ ID NO: 4 - protein, 24 aa, Homo sapiens
AKRHHGYKRKFHAKRHHGYKRKFH

SEQ ID NO: 5 - protein, 36 aa, Homo sapiens
AKRHHGYKRKFHAKRHHGYKRKFHAKRHHGYKRKFH

(19) United States
(12) Patent Application Publication
(10) Pub. No.: US 2017/0022256 A1
(43) Pub. Date: Jan. 26, 2017

(54) ANTI-FUNGAL AND ANTI-BACTERIAL PEPTIDE AND THERAPEUTIC METHOD USING SAME

(71) Applicant: GENERAL BIOLOGICALS CORPORATION, Hsinchu County (TW)

(72) Inventors: Wen-Chi Cheng, Hsinchu County (TW); Ming-Sun Liu, Hsinchu County (TW); Frank Lin, Hsinchu County (TW); Chung-Yu Lan, Hsinchu (TW); Guan-Yu Lin, Hsinchu (TW); Hsueh-Fen Chen, Hsinchu (TW)

(73) Assignee: GENERAL BIOLOGICALS CORPORATION, Hsinchu County (TW)

(21) Appl. No.: 15/175,011

(22) Filed: Jun. 6, 2016

Related U.S. Application Data
(60) Provisional application No. 62/196,292, filed on Jul. 23, 2015.

(51) Int. Cl. C07K 14/435

(52) U.S. Cl. CPC C07K 14/435; A61K 38/00

(57) ABSTRACT

The present invention provides an anti-bacterial peptide which has anti-fungal and anti-bacterial effect. The anti-bacterial peptide is a novel peptide sequence, wherein the peptide sequence comprises a sequence of at least two SEQ ID NO: 1. The present invention also provides a method for treating a subject infected with a fungus and a bacterium, which comprises providing the subject with an anti-bacterial peptide, wherein the anti-bacterial peptide comprises a sequence of at least two SEQ ID NO: 1.

### BACKGROUND OF THE INVENTION

The present invention provides an anti-fungal and antibacterial peptide characterized in that the anti-fungal and antibacterial peptide is a derivative of antibacterial peptide P-113.

### BACKGROUND OF THE INVENTION

Immunocompromised patients are susceptible to infections caused by Candida albicans which is a common opportunistic pathogen. Sometimes these infections cause fetal death. Candidate patients include patients suffering from Alzheimer's Disease, cancer patients receiving chemotherapy or radiotherapy, patients suffering from diabetes or xerostomia. Once infected, these patients are prone to candidiasi (oral thrush) or even develop systemic infection which, in turn, causes multiple organ failure. As to the treatment, Candida albicans are resistant to multiple antibiotics.

Histatins, a group of peptides abundant in histidine and found in saliva, are secreted by human parotid gland and submandibular gland. At present, about 12 histatins have been discovered. Histatin 1, histatin 3 and histatin 5 are the three major forms (constitute approximately 70-80% of total histatins), which are 38, 32, and 24 amino acid peptides, respectively, and have highly homologous sequence. Histatin 5 is proteolytically derived from histatin 3 and other histatins are proteolytically derived from these three major forms.

The three major histatins exhibit antimicrobial activities against a plurality of oral microbial infections. These histatins, secreted in human body, are capable of killing Candida albicans in both balstopore and mycelial forms and have antibacterial effect on a wide variety of bacteria, including Streptococcus mutans, Porphyromonas gingivalis, Actinomyces viscosus, etc.

Therefore, anti-bacterial substances produced by human body can provide effective treatment of microbial infections.

### DETAILED DESCRIPTION OF THE INVENTION

The present invention demonstrates that the antibacterial activity of the antibacterial peptide-113 (P-113) increases as the concentration or treating time increases and can effectively kill bacterial strains resistant to antibiotics. Examples of the present invention prove that P-113Du and P-113Tri (SEQ ID NOS: 4 and 5, respectively), which are derivatives of the peptide P-113, contain α-helix structure and exhibit more effective antibacterial activity in high-salt environment as compared to P-113. More importantly, P-113Du and P113Tri can kill suspension cells of Candida albicans more effectively than P-113. Accordingly, the present invention demonstrates that P-113 and antibacterial peptides derived from P-113 have a high potential for providing antibacterial activities.

The present invention provides P-113-derived antifungal or antibacterial peptides, which comprise P-113-HH, P-113-LL, P-113Du and P-113Tri. The amino acid sequence of P-113-HH is SEQ ID NO: 2 or its derivatives, the amino acid sequence of P-113-LL is SEQ ID NO: 3 or its derivatives, the amino acid sequence of P-113-Du is SEQ ID NO: 4 or its derivatives, and the amino acid sequence of P-113-Tri is SEQ ID NO: 5 or its derivatives.

The term “P-113” used herein is a peptide sequence which comprises SEQ ID NO: 1. P-113 (comprising SEQ ID NO: 1) and its derivatives further comprise a peptide sequence which modifies the amino acid sequence, for example: the C-terminus of the amino acid sequence is modified by adding NH₂ to the C-terminus. For example, the C-terminus of SEQ ID NO: 1 is modified with NH₂, more specifically, the carboxyl group of the last amino acid His of the amino acid sequence is modified by NH₂. The preparation of P-113 peptide structure can be found in U.S. Pat. Nos. 5,631,228, 5,646,119, 5,885,965 and 5,912,230, which are hereby incorporated by reference in their entireties.

The term “a” or “an” as used herein to describe the element and ingredient of the present invention may mean one or more. The term is used only for convenience and providing the basic concepts of the present invention. Furthermore, unless otherwise required by context, singular terms include pluralities and plural terms include the singular. When used in conjunction with the word “comprising” in a claim, the term “a” or “an” may mean one or more than one.

The term “or” as used herein may mean “and/or.”

The present invention provides a peptide which comprises a sequence of at least two SEQ ID NO: 1. In a preferred embodiment, the sequence of the at least two SEQ ID NO: 1 is SEQ ID NO: 4. In a more preferred embodiment, the sequence of the at least two SEQ ID NO: 1 is SEQ ID NO: 5. Therefore, the sequence of SEQ IQ NO: 4 is two consecutive sequences of SEQ ID NO: 1; and the sequence of SEQ ID NO: 5 is three consecutive sequences of SEQ ID NO: 1.

The term “peptide” as used herein may typically refer to a peptide shorter in length. Therefore, peptides, oligopeptides, dimers, multimers and the like are within the scope as defined. The definition intends to cover full-length proteins and fragments thereof. The term “polypeptide” and “protein” also includes post-expression modification of polypeptides and proteins, for example glycosylation, acetylation, phosphroylation and the like. For purposes of the present invention, “polypeptide” may include “modification” of a native sequence, such as deletion, insertion, substitution (the nature may be conservative or include the following substitution: any one of the 20 amino acids normally found in human proteins, or any other naturally or non-naturally occurring amino acids or atypical amino acids) and chemical modification (insertion of or substitution with mimetic peptides). These modifications may be deliberate or site-directed mutagenesis, or by chemically modifying amino acid to delete or connect chemical moieties, or may be accidental, for example, due to mutation induced by protein-generating hosts or due to mistakes caused by PCR amplifications.

A method for treating a subject infected by a bacterium or a fungus, comprising providing a composition to the subject, wherein the composition comprises an effective amount of a peptide and a pharmaceutically acceptable carrier, wherein the peptide comprises a sequence of at least two SEQ ID NO: 1.

Antibacterial peptide P-113, a histatin-5, consists of 12 amino acids which are the components of histatin-5. The P-113 comprises a sequence set forth in SEQ ID NO: 1. P-113Du comprises the sequence set forth in SEQ ID NO: 4 which is composed of two SEQ ID NO: 1 linked together. Therefore, in one embodiment the sequence of the at least two SEQ ID NO: 1 is SEQ ID NO: 4. P-113Du comprises the sequence set forth in SEQ ID NO: 5 which is composed of three SEQ ID NO: 1 linked together. Therefore, in one embodiment the sequence of the at least two SEQ ID NO: 1 is SEQ ID NO: 5.

In one embodiment, the peptide comprises a sequence of at least two SEQ ID NO: 1, wherein the content of the α-helical secondary structure contained in the sequence of the at least two SEQ ID NO: 1 is at least higher than 10%. In one preferred embodiment, the peptide comprises a sequence of at least two SEQ ID NO: 1, wherein the content of the α-helical secondary structure contained in the sequence of the at least two SEQ ID NO: 1 is at least higher then 15%. In one more preferred embodiment, the peptide comprises a sequence of at least two SEQ ID NO: 1, wherein the content of the α-helical secondary structure contained in the sequence of the at least two SEQ ID NO: 1 is at least higher then 20%.

In one embodiment, the anti-fungal and anti-bacterial refer to treating fungal infections and/or bacterial infections. The term “anti-fungal” as used herein includes anti-fungal properties of various forms, for example, inhibiting the growth of fungal cells, killing fungal cells, or interfering with or impeding fungal life cycles, such as spore germination, sporulation, mating. The term “anti-bacterial” as used herein includes killing bacteria, eliminating bacteria, disinfecting, inhibiting bacteria, anti-mildew or anti-mitotic.

The term “bacterium” or “fungus” as used herein includes: Candida albicans, Actinomyces actinomycetemcomitans, Actinomyces viscosus, Bacteroides forsythus, Bacteroides fragilis, Bacteroides gracilis, Bacteroides ureolyticus, Campylobacter concisus, Campylobacter rectus, Campylobacter showae, Campylobacter sputorum, Capnocytophaga gingivalis, Capnocytophaga ochracea, Capnocytophaga sputigena, Clostridium histolyticum, Eikenella corrodens, Eubacterium nodatum, Fusobacterium nucleatum, Fusobacterium periodonticum, Peptostreptococcus micros, Porphyromonas endodontali, Porphyromonas gingivalis, Prevotella intermedia, Prevotella nigrescens, Propionibacterium acnes, Pseudomonas aeruginosa, Selenomonas noxia, Staphylococcus aureus, Streptococcus constellatus, Streptococcus gordonii, Streptococcus intermedius, Streptococcus mutans, Streptococcus oxalis, Streptococcus pneumonia, Streptococcus sanguis, Treponema denticola, Treponema pectinovorum, Treponema socranskii, Veillonella parvula and Wolinella succinogenes.

In another embodiment, the fungus include a Candida spp. In a preferred embodiment, the Candida spp. includes infectious Candida, a Candida albicans, a C. tropicalis, a C. dubliniensis, a C. glabrata, a C. guilliermondii, a C. krusei, a C. lusitaniae, a C. parapsilosis, a C. pseudotropicalis, and a Candida famata. In a more preferred embodiment, the fungus is a Candida albicans.

In one embodiment, the fungus includes a fungus having drug resistance. In a preferred embodiment, the Candida is a Candida having drug resistance. In a more preferred embodiment, the Candida is a Candida albicans having drug resistance. In an even more preferred embodiment, the drug resistance includes resistance to fluconazole, amphoterincin B and caspofungin.

In another embodiment, the at least two SEQ ID NO: 1 (i.e., SEQ ID NOS: 4 or 5) maintain anti-fungal or anti-bacterial activity in a high salt environment. Thus, P-113Du (SEQ ID NO: 4) and P-113Tri (SEQ ID NO: 5) exhibit better environmental endurance than P-113 did, i.e., the stability of peptide sequence is better than that of P-113.

In one embodiment, the at least two SEQ ID NO: 1 (i.e., SEQ ID NOS: 4 or 5) exhibit inhibitory effect on fungal growth at a pH value ranging from 4 to 9.

In another embodiment, the composition further destructs and kills biofilms formed by a bacterium or a fungus. In a preferred embodiment, the composition further treats a subject infected with a biofilm.

In another embodiment, the bacterium includes Pseudomonas aeruginosa, Klebsiella pneumoniae, Enterobacter aerogenes, and Staphylococcus aureus.

The term “pharmaceutically acceptable carrier” as used herein is determined by specific combination and specific method the composition is administered. The term “carrier” as used herein includes any and all solvents, dispersing media, vehicles, coatings, diluents, anti-bacterial and anti-fungal agents, penetration and absorption delaying agents, buffers, carrier solutions, suspension fluids, colloidal gels, etc. These media and reagents are used as pharmaceutically active ingredients, which is well-known in the art. If a conventional medium or reagent is incompatible with any active ingredients, care must be taken when it is used in a composition for treatment purposes. Complementary active ingredients may also be incorporated into the composition. The term “pharmaceutically acceptable” as used herein refers to molecular entities and compositions administered to a subject without causing any allergic reactions or similar negative effects. It is conventional and well known in the art to use proteins as active ingredients in water compositions. Typically, the composition is prepared as liquid solution or suspension for injections, or prepared in solid form which is soluble or suspendable for injections.

The term “effective amount” as used herein refers to a therapeutic dose which can prevent, reduce, impede or reverse the development of a symptom in a subject, or can partially, completely alleviate a symptom which exists when the subject begins receiving the treatment.

In one embodiment, the effective amount of the peptide ranges from 0.01 μg/ml to 200 μg/ml. In one preferred embodiment, the effective amount of the peptide ranges from 0.1 μg/ml to 50 μg/ml. In a more preferred embodiment, the effective amount of the peptide ranges from 0.1 μg/ml to 20 μg/ml.

In one embodiment, the subject is an animal, preferably a mammal, more preferably a human.

The peptide (comprising at least two SEQ ID NO: 1) and the pharmaceutically acceptable carrier may be administered to a subject through a number of different routes known in the art. In one embodiment, the peptide sequence (comprising at least two SEQ ID NO: 1) and the pharmaceutically carrier are administered intravenously, via muscle, subcutaneously, topically, orally or via inhalation. The drug is delivered to target sites via the digestive system and the circulatory system.

The peptide (comprising at least two SEQ ID NO: 1) and the pharmaceutically acceptable carrier may be prepared by a sterile aqueous solution or a dispersion, an aqueous suspension, an oil emulsion, water in a water-in-oil emulsion, site-specific emulsion, a sustained-release emulsion, a viscous emulsion, a micro-emulsion, a nano-emulsion, a liposome, microparticle s, microspheres, nanospheres, nano-particles, micro-mercury and several sustained-release natural or synthetic polymers. The pharmaceutically acceptable carrier and P-113 modified peptide may be prepared as aerosols, tablets, pills, sterile powders, suppositories, lotions, creams, ointments, pastes, gels, hydrogels, sustained delivery devices, or other formulations which may be used for drug delivery.

### BRIEF DESCRIPTION OF THE DRAWINGS

FIG. 1 shows that as the concentration and treating time increase the anti-bacterial activity of peptide P-113 against Candida albicans also increases. The experiment is conducted by treating Candida albicans cell suspension at 37° C. with different concentrations of peptide P-113 for different period of time. Results represent the average of three independent experiments.

FIG. 2 shows that anti-bacterial peptide P-113 exhibits anti-bacterial effect on a number of clinically drug-resistant Candida. FIG. 2(A) to FIG. 2(C) show that P-113 exhibits anti-bacterial effect on clinically isolated bacterial strains having drug resistance to fluconazole, amphoterincin B, or caspofungin, respectively, after being treated with anti-bacterial peptide P-113. (ATCC: source of standard strains).

FIG. 3 shows helical-wheel projections of P-113 and anti-bacterial peptides derived from P-113. Different shapes represent amino acids having different properties, round, diamond, triangle, and pentagon represent hydrophilic, hydrophobic, positively charged and negatively charged amino acids, respectively. In addition, color green, yellow, red and blue represent hydrophobic, lowly hydrophilic, highly hydrophilic and electrically charged amino acids, respectively.

FIG. 4 shows the secondary structure of P-113 and its peptide derivatives P-113Du and P-113Tri measured in 85% trifluoroethanol solution (trifluoroethanol; TFE; pH 6.0) at 25° C. by a Circular Dichoism Spectrometer. P-113, P-113Du and P-113Tri are analyzed over the wavelength range of 195-260 nm with readings every lnm to obtain the mean residue molar ellipticity (θ). P-113, P-113Du and P-113Tri all have positive response at 195 nm and two negative responses at 208 and 222 nm, which shows that the α-helical secondary structures have been generated. Based on BeStSel analysis, the α-helical structure content of P-113 is 2.9%, the α-helical structure content of P-113Du and P-113Tri are 10.6% and 21.4%, respectively. The higher the content is, the more stable the α-helical structure is. P-113Tri has the most obvious and the stablest α-helical structure, which can bind with bacterial cell membrane tightly and provides a strong anti-bacterial effect. Accordingly, P-113Tri has the best anti-bacterial effect.

FIG. 5 show the effect of salt concentration and pH value on P-113 and its derivative peptides P-113Du and P-113Tri. FIG. 5(A) shows that P-113, P-113Du and P-113Tri are dissolved in different concentration (12.5, 62.5 and 93.75 mM) of sodium acetate solution (NaOAc) and Candida albicans is treated with different concentrations of P-113, P-113Du and P-113Tri at 37° C. for one hour. FIG. 5(B) shows the results of Candida albicans after being treated at different pH values and then cultivated in YPD medium for 1 day. Different concentrations of anti-bacterial peptides are represented by numbers shown in the right box.

FIG. 6 shows the anti-bacterial activities of P-113 and its derivative peptides P-113Du and P-113Tri against Candida albicans cell suspension. Candida albicans is treated with different concentrations of P-113, P-113Du and P-113Tri at 37° C. for 1 hour. Results represent the average of three independent experiments.

FIG. 7 shows the effect of P-113 and its derivative peptides on Candida albicans biofilms. FIG. 7(A) shows the effect of anti-bacterial peptides P-113, P-113Du and P-113Tri on Candida albicans biofilms. The results of XTT reduction assays show that the Candida albicans biofilm is highly sensitive to P-113Tri. In FIG. 7(B), the effect of P-113 and its derivative peptides on the surface of a Candida albicans biofilm is observed by a scanning electron microscopy (SEM), a protruding tumor-like rough surface is found after being treated with anti-bacterial peptides, which is similar to the effect of generating peroxide radicals. The Candida albicans biofilm is then treated with L-ascorbic acid, the rough surface disappears, which shows a phenomenon of compensation.

FIG. 8 shows the results of the effects of anti-bacterial peptides P-113, P-113Du and P-113Tri on Candida albicans compensated with L-ascorbic acid. The effect of the anti-bacterial peptide can be compensated by adding L-ascorbic acid which significantly decreases the anti-bacterial effect.

### EXAMPLES

The following examples are non-limiting and are merely representative of various aspects and features of the present invention.

### Example 1

### Preparation of P-113, P-113 Derivatives, Modified P-113 and Derivative Peptides

Peptide-113 (P-113) originates from histatin-5. P-113 comprises 12 functional amino acid fragments from histatin-5 and the amino acid sequence of P-113 is set forth in SEQ ID NO: 1. The preparation may refer to U.S. Pat. Nos. 5,631,228, 5,646,119, 5,885,965, and 5,912,230, all of which are hereby incorporated by reference in their entireties.

The NH₂-end on the C-terminus of P-113, which had 12 amino acids, was modified by using a peptide synthesizer. P-113 was synthesized by standard Fmoc-based solid-phase peptide synthesis and prepared in the peptide synthesizer. The synthesized peptide was purified by reversed phase high performance liquid chromatography (RP-HPLC). After purification, the present invention employed two yeast systems, peptidylglycine alpha-monooxygenase (PAM) and peptidylamidoglycolate lyase (PGL) to seal the amino group on the C-terminus of P-113. Monooxygenase was first catalyzed to form alpha-hydroxyglycine derivatives which were glycine-extended precursors, PAM product was then catalyzed by lyase to form amidated peptide and glyoxylate via degradation.

Modified P-113 peptides were synthesized by chemical reactions using P-113 as the basis or prepared by a recombinant DNA comprising mutated nucleic acid sequences. Four modified P-113 peptides were prepared in the present invention: P-113-HH (SEQ ID NO: 2), P-113-LL (SEQ ID NO: 3), P-113Du (SEQ ID NO: 4) and P-113Tri (SEQ ID NO: 5).

### Example 2

### Time-Dependent and Concentration Dependent Antibacterial Activity of P-113 Against Candida

Method:

Candida albicans strain SC5314 (wild type, WT) was cultivated in Yeast extract Peptone Dextrose medium (YPD medium) at 30° C. overnight and then transferred to 5 ml of fresh YPD culture broth, subjected to cultivation for another 5 hours. After the bacteria were collected by centrifugation, washed the bacteria with 12.5 mM sodium acetate (NaOAc) twice, redissolved in each well of a 96-well plate with 12.5 mM NaOAc (1.5×10⁶ cells in 0.1 ml of 12.5 mM NaOAc). Then, the bacteria were treated with different concentrations of P-113 for different reaction time at 37° C. 3.98 ml of Phosphate-buffered saline (PBS) was added into each well, took 25 μl of bacterial liquid out and smeared on a solid YPD medium, after being cultivated at 30° C. for 24 hours, the number of colonies were counted.

Results:

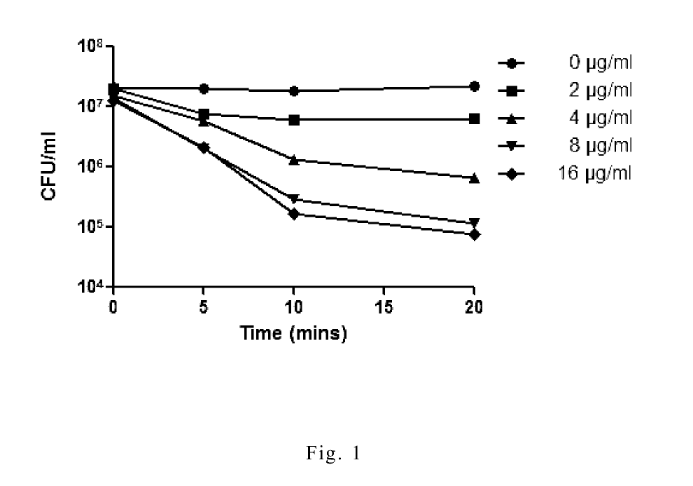

As shown in FIG. 1, the cellular survival rate decreased, which correlated to an increased concentration of P-113 and a prolonged co-cultivation time. Therefore, the anti-bacterial activity of P-113 against Candida was time-dependent as well as concentration-dependent.

### Example 3

### P-113 was Effective Against Clinical Isolates of Drug-Resistant Candida

Method:

The present invention examined the effect of P-113 on the activities of 34 clinical isolates of Candida (see Table 1). After clinical isolates were inoculated in YPD cultural broth (1% yeast extract, 2% peptone and 2% dextrose) and subjected to overnight shaking cultivation at 30° C., the bacterial cells were collected by centrifugation, washed with YPD and then cultivated in YPD cultural broth (initial optical density at 600 nm [OD ₆₀₀]˜0.5) allowing to grow for 5 hours. To test the sensitivity of bacterial cells to anti-fungal drugs, the cells were inoculated in a solid YPD medium which contained fluconazole (300 μg/ml). To test the effect of P-113, cells were collected by centrifugation, washed with PBS and redissolved in 12.5 mM NaOAc, the concentration of the cells was adjusted to ˜0.1[OD₆₀₀]/ml, and then treated with P-113. The mixture solution was subjected to shaking cultivation for 1 hour at 37° C. and under 5% CO₂, inoculated on the solid YPD medium and cultivated for 2 days.

TABLE 1
Clinical isolates of Candida

| No. | Species |
|---|---|
| 1 | unknown |
| 2 | unknown |
| 3 | C. albicans |
| 4 | C. albicans |
| 5 | unknown |
| 6 | C. tropicalis |
| 7 | C. tropicalis |
| 8 | C. albicans |
| 9 | C. famata |
| 10 | C. albicans |
| 11 | C. albicans |
| 12 | C. tropicalis |
| 13 | C. tropicalis |
| 14 | C. tropicalis |
| 15 | unknown |
| 16 | C. albicans |
| 17 | C. glabrata |
| 18 | C. albicans |
| 19 | C. tropicalis |
| 20 | C. albicans |
| 21 | C. albicans |
| 22 | C. albicans |
| 23 | C. albicans |
| 24 | unknown |
| 25 | C. albicans |
| 26 | C. albicans |
| 27 | C. albicans |
| 28 | unknown |
| 29 | C. albicans |
| 30 | C. albicans |
| 31 | C. albicans |
| 32 | C. albicans |
| 33 | C. albicans |
| 34 | C. albicans |

Results:

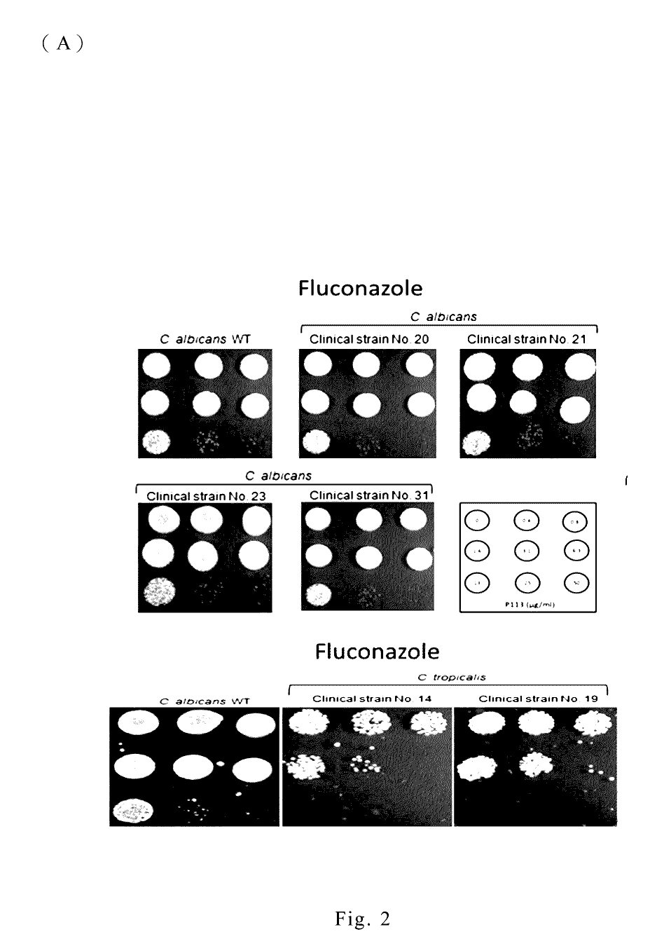

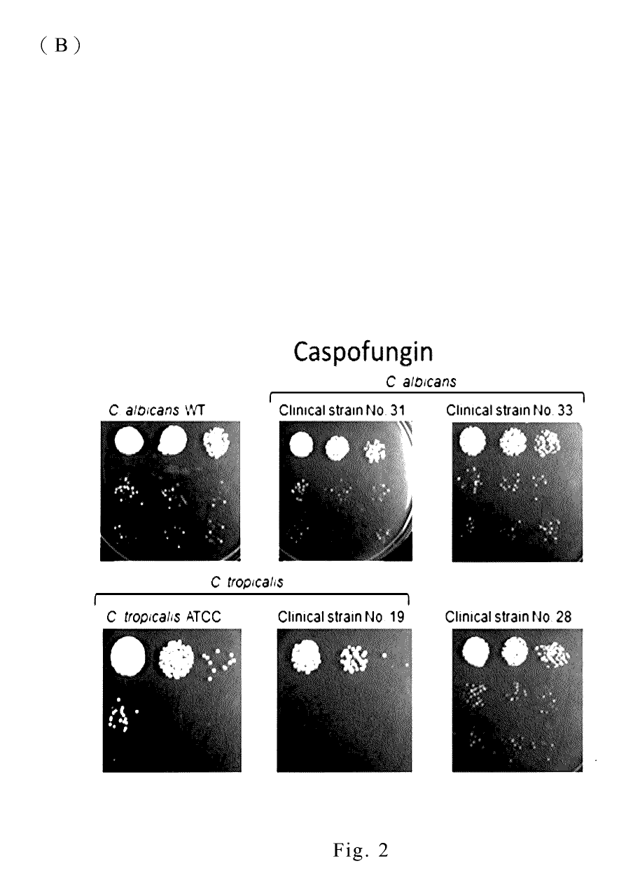

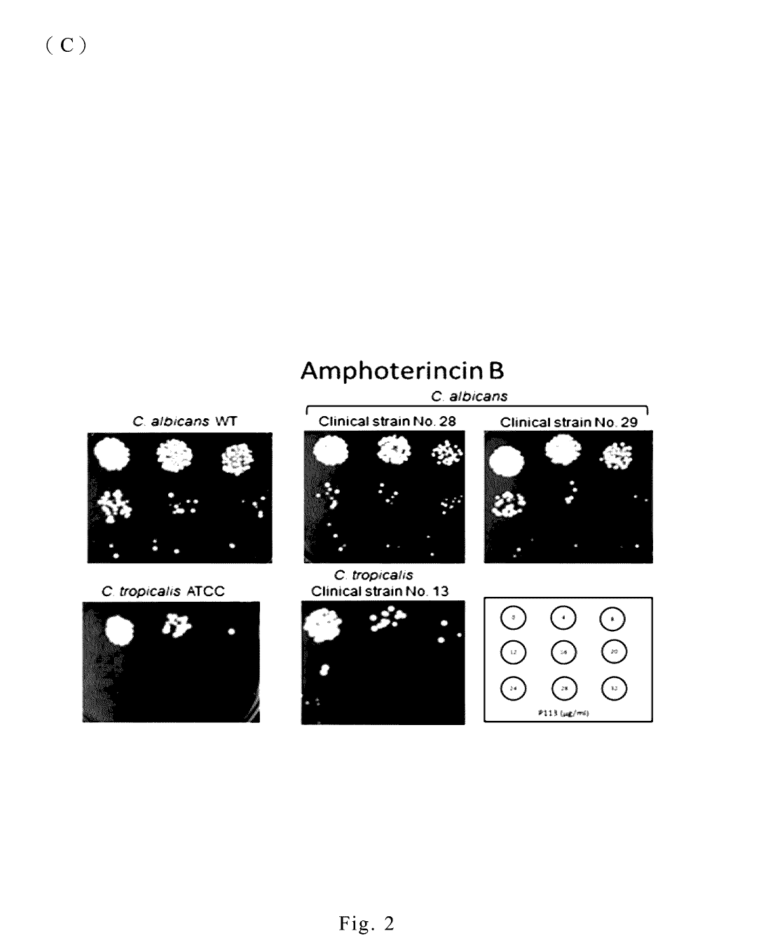

FIGS. 2(A) to 2(C) show the results of clinical isolates of Candida treated with anti-fungal drugs. The present invention found that, out of 34 clinical isolates of Candida, 6 of them were fluconazole-resistant strains (No. 14, 19, 20, 21, 23, 31); 5 of them were amphoterincin B-resistant strains (No. 9, 13, 25, 28, 29); and 4 of them were caspofungin-resistant stains (No. 19, 28, 31, 33). P-113 exhibited inhibitory effect on these clinical isolates of drug-resistant Candida strains.

### Example 4

### Characteristics of P-113 Peptide Derivatives

Method:

By altering the sequence characteristics of P-113, the present invention designed and synthesized a number of different P-113 peptide derivatives which were imparted with an improved anti-bacterial activity against Candida. The ratio of hydrophobic amino acids and net electrical charge of these derivatives were predicted by an Antimicrobial Peptide Database (APD). The helical wheel representing the proteins (http://aps.unmc.edu/AP/main.php) was made by using helical wheel projections (http://rzlab.ucr.edu/scripts/wheel/wheel.cgi).

Results:

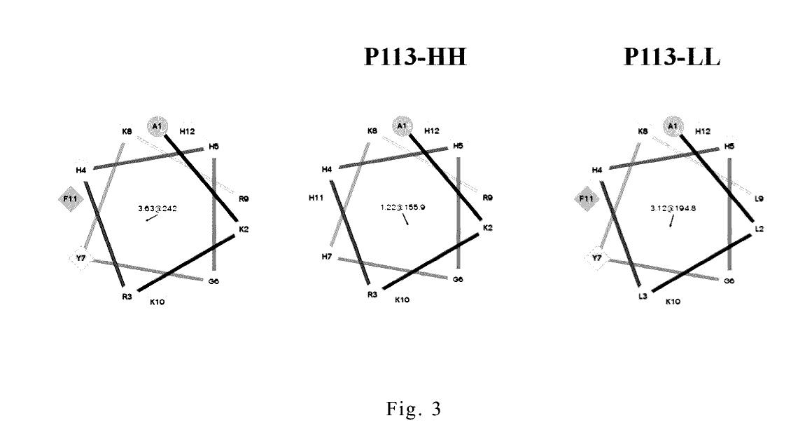

The results were shown in Table 2. To enhance the anti-bacterial activity of P-113 against Candida albicans, the present invention synthesized P-113 derivatives and tested their anti-fungal or anti-bacterial activities against Candida. The results showed that P-113-HH had higher hydrophobicity and lower amphiphilic properties than P-113 did, but P-113-LL had higher hydrophobicity and higher amphiphilic properties than P-113 did. In addition, P-113Du and P-113Tri carried higher positive valence than P-113 did. FIG. 3 shows helical wheels of P-113 and its derivatives.

TABLE 2
Sequences and characteristics of P-113
peptide and its derivatives

| | | Hydrophobicity | Positive |
|---|---|---|---|
| Peptide | Sequence | ratio | charge |
| P-113 | AKRHHGYKRKFH | 16% | +5 |
| P113-RR | AKRHHGHKRKHH | 8% | +5 |
| P113-LL | ALLHHGYKLKFH | 41% | +2 |
| P-113Du | AKRHHGYKRKFH | 16% | +10 |
| | AKRHHGYKRKFH | | |
| P-113Tri | AKRHHGYKRKFH | 16% | +15 |
| | AKRHHGYKRKFH | | |
| | AKRHHGYKRKFH | | |

### Example 5

### Structure of P-113 and its Derivatives P-113

Method:

Circular dichoism spectra of P-113 and its derivatives were recorded at 25° C. over the wavelength range of 195-260 nm with readings every 1 nm by using a Circular Dichoism Spectrometer (AVIV Company) and a quartz cuvette having 1 mm optical path length.

Ellipticities were represented as mean residue molar ellipticity (MRE). P-113, P-113Du and P-113Tri were dissolved in 85% trifluoroethanol (TFE).

Results:

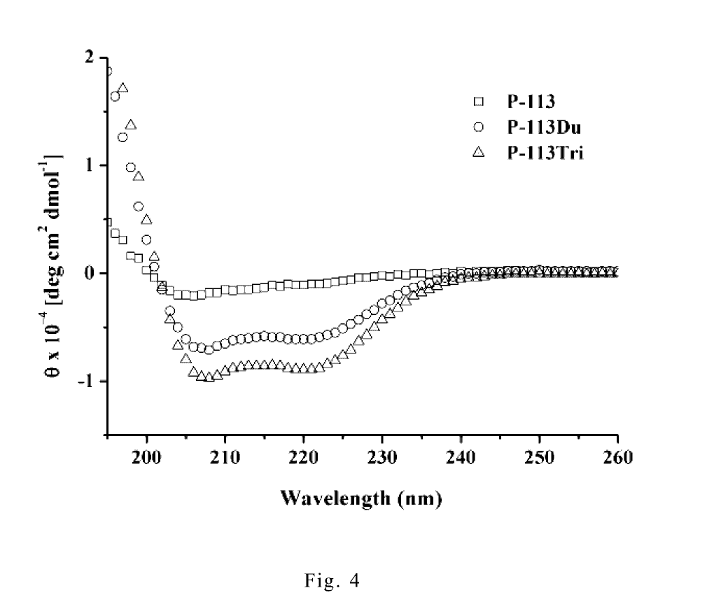

As shown in FIG. 4, P-113, P-113Du and P-113Tri all had α-helical structures.

### Example 6

### Salt Endurance of P-113 and its Derivatives

Method:

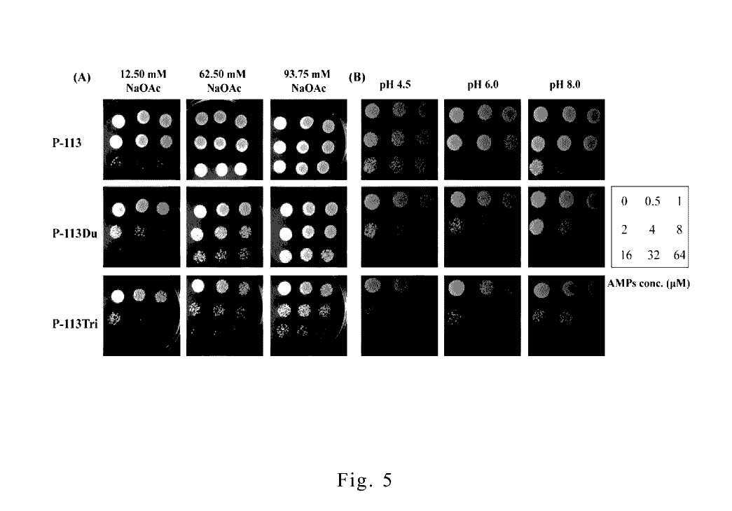

Wild type (WT) Candida albicans was cultivated in YPD medium at 30° C. overnight and then transferred to 5 ml of fresh YPD culture broth, subjected to cultivation for another 5 hours. After the bacteria were collected by centrifugation, washed the bacteria with 12.5 mM sodium acetate twice, redissolved with 12.5 mM NaOAc to yield a bacterial concentration of 1.5×10⁶ cells/ml. 50 μl of bacterial liquid was taken out to mix with 50 μl of sequence-diluted anti-bacterial peptides and placed in different wells of a 96-well plate for 1 hour reaction at 37° C. (as shown in FIG. 5(A). Then, 50 μl of bacterial liquid mixture was taken out and added into 450 μl PBS to terminate the reaction. Finally, 25 μl was taken out and smeared on a solid YPD medium.

Results:

The results in FIG. 5(A) showed that P-113Tri had strong anti-bacterial activity in high salt environment (62.5 and 93.75 mM), P-113Du had anti-bacterial activity but P-113 lost its anti-bacterial activity in high salt environment. The results in FIG. 5(B) showed that P-113 exhibited the best anti-bacterial activity at pH 6.0 and achieved complete sterilization when the concentration of P-113 was 16 μg/ml. To achieve complete sterilization at pH 8.0, the concentration must be increased to 64 μg/ml; at pH 4.5, P-113 did not exhibit any anti-bacterial activities even when the concentration was as high as 64 μg/ml. However, P-113Du and P-113Tri exhibited good anti-bacterial activities at pH 6.0, exhibited inhibitory effect when the concentration was 4 μg/ml, at pH 8.0 or pH 4.5, weak alkaline or weak acidic environment, they achieved complete sterilization against Candida albicans when the concentration was 8 μg/ml.

### Example 7

### Anti-Bacterial Activities of P-113 and its Derivatives

(A) Anti-Bacterial Activities of P-113 and its Derivatives Against Candida albicans

Method:

Wild type (WT) Candida albicans was cultivated in YPD medium at 30° C. overnight and then transferred to 5 ml of fresh YPD culture broth, subjected to cultivation for another 5 hours. After the bacteria were collected by centrifugation, washed the bacteria with 12.5 mM sodium acetate twice, redissolved with 12.5 mM sodium acetate to yield a bacterial concentration of 1.5×10⁶ cells/ml. 50 μl of bacterial liquid was taken out to mix with 50 μl of sequence-diluted anti-bacterial peptides and placed in different wells of a 96-well plate to react at 37° C. for 1 hour (as shown in FIG. 5(A)). Then, 20 μl of bacterial liquid mixture was added into 780 μl PBS to terminate the reaction. Finally, 50 μl was taken out and smeared on a solid YPD medium, cultivated at 30° C. for 24 hours, then the number of colonies were counted.

Results:

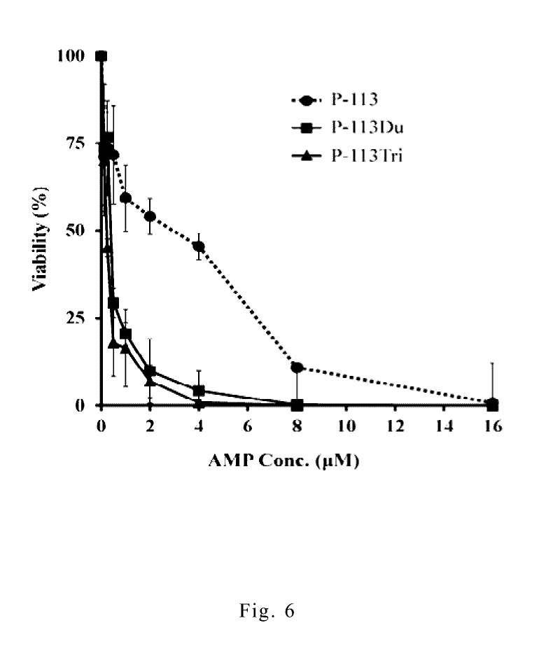

As shown in FIG. 6, P-113Tri and P-113Du exhibited better anti-bacterial activities than P-113 did.

(B) Anti-Bacterial Activities of P-113 and its Derivatives Against Candida albicans Biofilms.

Methods:

Candida albicans strain SC5314 was cultivated in YPD medium overnight and then transferred to fresh YPD culture broth, diluted until the concentration was 3×10⁵ cells/ml. 100 μl of bacterial liquid was placed in a 96-well plate for cultivation at 37° C. for 24 hours, the formed biofilms were washed with sodium acetate (12.5 mM). Then, sequence-diluted anti-bacterial peptides P113, P-113 dimer and P-113Tri (0-200 μM) were added, reacted at 37° C. for 1 hour, washed with PBS twice. Cellular activities of the biofilms were determined by using XTT (2,3-bis (2-methoxy-4-nitro-5-sulfophenyl)-2H-tetrazolium-5-carboxanilide) reduction assays to analyze the cellular survival rate. To perform the reaction, XTT (0.5 mg/ml) and Menadione (0.5 μM) were dissolved in PBS and added into a 96-well plat form by biofilms, reacted at 30° C. for 30 minutes, the optical density was measured at wavelength 490 inn (OD₄₉₀).

Results:

The results indicated that P-113Tri exhibited the best anti-bacterial activity against biofilms.

### Example 7

(A) Method: Bacterial biofilms were cultivated in a multi-well plate. After P-113, P113Du and P-113Tri were added, the biofilms were observed by using a scanning electron microscopy.

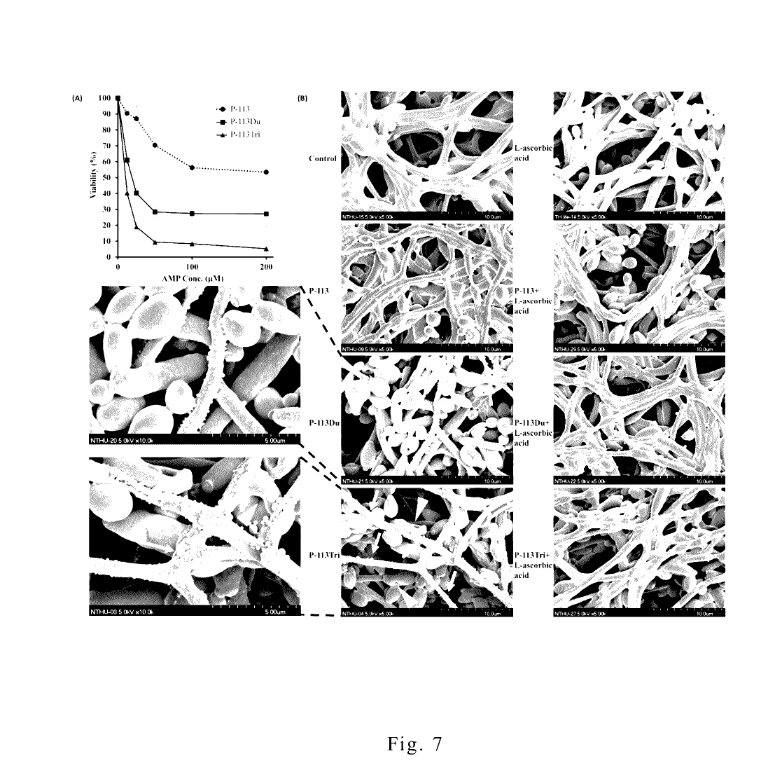

Results: FIG. 7 were biofilm morphology. 50 μM peptide was added to bacterial biofilms, the morphology was observed by a scanning electron microscopy, magnified 5000 times. For those biofilms added with P-113Du and P-113Tri, in order to observe the morpholy these biofilms were further magnified 10000 times. Rough surface caused by P-113Du and P-113Tri disappeared when 1 M L-ascorbic acid was added.

(B) L-ascorbic acid compensation method: Candida albicans strain SC5314 was cultivated in YPD medium overnight and then transferred to fresh YPD culture broth, diluted until the concentration was 3×10⁵ cells/ml. 100 μl of bacterial liquid was placed in a 96-well plate, cultivated at 37° C. for 24 hours, the formed biofilms were washed with sodium acetate (12.5 mM). Then, sequence-diluted anti-bacterial peptides P113, P-113 dimer and P-113Tri (0 μM-200 μM) were added, reacted at 37° C. for 1 hour, washed with PBS twice. Cellular activities of the biofilms were determined by using XTT (2,3-bis (2-methoxy-4-nitro-5-sulfophenyl)-2H-tetrazolium-5-carboxanilide) reduction assays to analyze the cellular survival rate. To perform the reaction, XTT (0.5 mg/ml) and Menadione (0.5 μM) were dissolved in PBS and added into a 96-well plat form by biofilms, reacted at 30° C. for 30 minutes, the optical density was measured at wavelength 490 nm (OD₄₉₀). The cellular activity of the biofilm was represented as 100%.

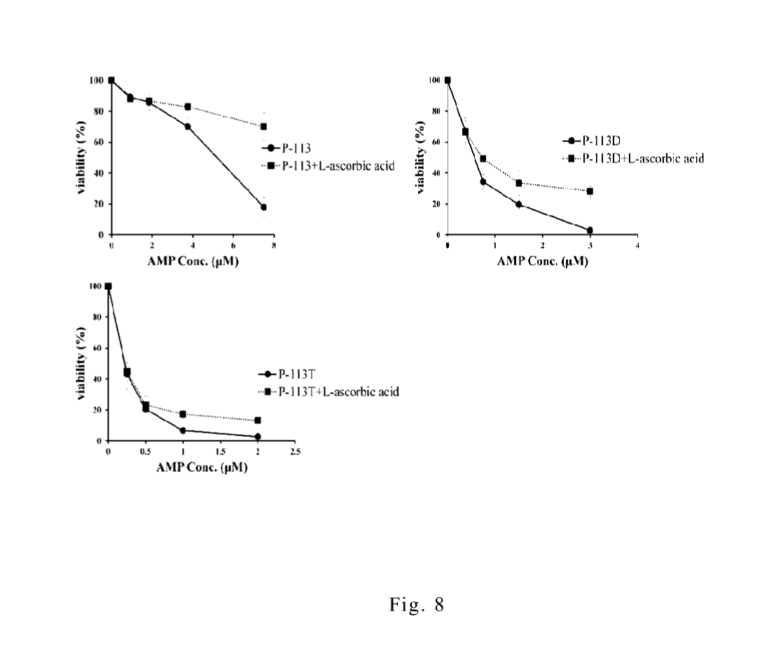

Results: As shown in FIG. 8, the anti-bacterial activities of P-113, P-113Du and P-113Tri against suspension cells were affected by L-ascorbic acid. Different concentrations of peptides and 50 mM L-ascorbic acid were added or not added to biofilms and anti-bacterial activities were observed. The mixture was allowed to stand at 37° C. for 1 hour. Cellular survival rate was determined by using XTT assays. Results represent the average of three independent experiments.

### Example 8

TABLE 3
Anti-bacterial effect of P-113Du and P-113Tri against
bacteria.

| | | MIC (μg/ml) in LYM | |
|---|---|---|---|
| | | medium for 20 hrs | |
| Bacterium | Bacterium | P-113Du | P-113Tri |
| Pseudomonas aeruginosa | PAO1, ATCC 15692 | 3.125 | 3.125 |
| Klebsiella pneumoniae | CG43 | 12.5 | 6.25 |
| Enterobacter aerogenes | ATCC 13048 | 25 | 12.5 |
| Staphylococcus aureus | ATCC 33591 | 25 | 25 |

Method: Wild type (WT) Pseudomonas aeruginosa, Klebsiella pneumoniae, Enterobacter aerogenes, Staphylococcus aureus were subjected to overnight shaking cultivation in LB, then transferred to fresh LB culture broth, subjected to cultivation for another 3 hours. The bacteria were collected by centrifugation, washed with sodium acetate (12.5 mM) twice, redissolved with 12.5 mM until the concentration was 1.5×10⁵ cells/ml. Mixed with sequence-diluted anti-bacterial peptides, placed in different wells of a 96-well place and reacted for 1 hour. Then 20 μl of bacteria liquid mixture was taken out to mix with 780 μl of PBS (Phosphate-buffered saline) to terminate the reaction. 50 μl of was taken out and smeared on a solid medium, after being cultivated at 30° C. for 24 hours, observed the formed colonies.

Results: As shown in the following table, P-113Du and P113Tri were able to effectively inhibit the growth of Pseudomonas aeruginosa, Klebsiella pneumoniae, Enterobacter aerogenes, and Staphylococcus aureus.

Table 3. Anti-Bacterial Effect of P-113Du and P-113Tri Against Bacteria.

The present invention illustratively described herein may suitably be practiced in the absence of any element of elements, limitation or limitations, not specifically disclosed herein. The terms and expressions employed herein have been used as terms of description and not of limitation, and there is no intention in the use of such terms and expressions of excluding any equivalents of the features shown and describes or portions thereof, but it is recognized that various modifications are possible within the scope of the invention claimed. Thus, it should be understood that although the present invention has been specifically disclosed by embodiments and examples, modifications and variations of the inventions embodied therein my be resorted to by those skilled in the art, and that such modifications and variations are considered to be within the scope of this invention.

What is claimed is:

1. A peptide, which comprises a sequence of at least two SEQ ID NO: 1.

2. The peptide of claim 1, wherein the sequence of the at least two SEQ ID NO: 1 is SEQ ID NO: 4.

3. The peptide of claim 1, wherein the sequence of the at least two SEQ ID NO: 1 is SEQ ID NO: 5.

4. The peptide of claim 1, wherein the content of α-helical secondary structure contained in the at least two SEQ ID NO: 1 is at least higher than 10%.

5. A method for treating a subject infected with a bacterium or a fungus, comprising providing a composition to the subject, wherein the composition comprises an effective amount of peptide sequence and a pharmaceutically acceptable carrier, wherein the peptide sequence comprises a sequence of at least two SEQ ID NO: 1.

6. The method of claim 5, wherein the sequence of the at least two SEQ ID NO: 1 is SEQ ID NO: 4.

7. The method of claim 5, wherein the sequence of the at least two SEQ ID NO: 1 is SEQ ID NO: 5.

8. The method of claim 5, wherein the content of α-helical secondary structure contained in the at least two SEQ ID NO: 1 is at least higher than 10%.

9. The method of claim 5, wherein the fungus is a Candida.

10. The method of claim 9, wherein the fungus is a Candida albicans.

11. The method of claim 9, wherein the fungus is a Candida which has drug resistance.

12. The method of claim 11, wherein the drug resistance is resistance to fluconazole, resistance to amphoterincin or resistance to caspofungin.

13. The method of claim 5, wherein the bacterium is Pseudomonas aeruginosa, Klebsiella pneumoniae, Enterobacter aerogenes, or Staphylococcus aureus.

14. The method of claim 5, wherein the effective amount ranges from 1 μg to 20 μg.
